(19) United States
(12) Patent Application Publication
(10) Pub. No.: US 2015/0353448 A1
(43) Pub. Date: Dec. 10, 2015

(54) PROCESS FOR THE SELECTIVE HYDROGENATION OF ACETYLENE TO ETHYLENE

(71) Applicant: UOP LLC, Des Plaines, IL (US)

(72) Inventors: Timur Voskoboynikov, Arlington Heights, IL (US); Vincent Mezera, Brookfield, IL (US); Laura E. Leonard, Western Springs, IL (US); Paul T. Barger, Arlington Heights, IL (US); Clayton Colin Sadler, Arlington Heights, IL (US); Michael Roy Smith, Rolling Meadows, IL (US); Andrea G. Bozzano, Northbrook, IL (US)

(21) Appl. No.: 14/298,041

(22) Filed: Jun. 6, 2014

(51) Int. Cl. C07C 5/09

(52) U.S. Cl. CPC C07C 5/09

(57) ABSTRACT

A process for a liquid phase selective hydrogenation of acetylene to ethylene in a reaction zone. In order to decrease the selectivity to C₄+ hydrocarbons, the concentration of acetylene in solvent is lowered by recycling solvent, using a split feed injection, or both. The streams can be split in to equal or unequal portions. A hot separator may be used to separate solvent from the reactor effluent, and the solvent may be recovered and used to decrease the concentration of acetylene in the solvent.

### FIELD OF THE INVENTION

This invention relates generally to processes for selectively converting alkynes to olefins, and more specifically for the selective hydrogenation of acetylene to ethylene.

### BACKGROUND OF THE INVENTION

Light olefin materials, including ethylene and propylene, represent a large portion of the worldwide demand in the petrochemical industry. Light olefins are used in the production of numerous chemical products, via polymerization, oligomerization, alkylation and other well-known chemical reactions. These light olefins are essential building blocks for the modern petrochemical and chemical industries for the production of items such as polyethylene. Producing large quantities of light olefin material in an economical manner, therefore, is a focus in the petrochemical industry.

The production of light olefins, and in particular ethylene, can be through steam or catalytic cracking processes. The cracking processes take larger hydrocarbons, such as paraffins, and convert the larger hydrocarbons to smaller hydrocarbons products. The primary product is ethylene. However, there are numerous other chemicals produced in the process. Among the many byproducts are hydrogen, methane, acetylene, and ethane.

Historically, naphtha cracking has provided the largest source of ethylene, followed by ethane and propane pyrolysis, cracking, or dehydrogenation. Due to the large demand for ethylene and other light olefinic materials, however, the cost of these traditional feeds has steadily increased.

Energy consumption is another cost factor impacting the pyrolytic production of chemical products from various feedstocks. Over the past several decades, there have been significant improvements in the efficiency of the pyrolysis process that have reduced the costs of production.

More recent attempts to decrease light olefin production costs include utilizing alternative processes and/or feed streams. In one approach, hydrocarbon oxygenates and more specifically methanol or dimethylether (DME) are used as an alternative feedstock for producing light olefin products. Oxygenates can be produced from available materials such as coal, natural gas, recycled plastics, various carbon waste streams from industry and various products and by-products from the agricultural industry. Making methanol and other oxygenates from these types of raw materials is well established and typically includes one or more generally known processes such as the manufacture of synthesis gas using a nickel or cobalt catalyst in a steam reforming step followed by a methanol synthesis step at relatively high pressure using a copper-based catalyst.

Once oxygenates are formed, the process includes catalytically converting oxygenates, such as methanol, into the desired light olefin products in an oxygenate to olefin (OTO) process. Techniques for converting oxygenates, such as methanol to light olefins (MTO), are described in U.S. Pat. No. 4,387,263, which discloses a process that utilizes a catalytic conversion zone containing a zeolitic type catalyst. This indirect route of production is often associated with energy and cost penalties, often reducing the advantage gained by using a less expensive feed material.

Another alternative process used to produce ethylene involves using pyrolysis to convert natural gas to ethylene. U.S. Pat. No. 7,183,451 discloses heating natural gas to a temperature at which a fraction is converted to hydrogen and a hydrocarbon product such as acetylene or ethylene. The product stream is then quenched to stop further reaction and subsequently reacted in the presence of a catalyst to form liquids to be transported.

A similar process is disclosed in U.S. Pat. No. 7,208,647 in which natural gas is combusted under suitable conditions to convert the natural gas into primarily ethylene and acetylene. The acetylene in the gaseous product stream is separated from the remaining products and converted to ethylene.

More recent efforts have focused on the use of supersonic reactors for the pyrolysis of natural gas into acetylene. For example U.S. Pat. Pub. No. 2014/0058149 discloses a reactor in which a fuel is combusted and accelerated to a supersonic speed. Natural gas is injected into the reactor downstream of the supersonic combustion gas stream, and the natural gas is converted into acetylene as an intermediary product. The reaction is quenched with a liquid to stop the reaction and the acetylene may be converted to the desired product ethylene in a hydrogenation zone.

Whether an undesired byproduct or one of the desired products, acetylene will irreversibly bond with many downstream catalysts, in particular with polymerization catalysts. Therefore, the production streams which include acetylene must be treated to remove or reduce the amount of acetylene. Additionally, in those processes that produce acetylene as an intermediary product, the majority of the acetylene must be converted to ethylene. One method of converting or reducing the amount of acetylene is selective hydrogenation.

Selective hydrogenation process can be utilized to reduce the acetylene concentration to a sufficiently low level and can be done in either a gas phase or a liquid phase. Since selective hydrogenation is a highly exothermic reaction, the liquid phase is sometimes preferred as it can better control temperature of the reaction. For example, U.S. Pat. No. 8,460,937 discloses a process in which acetylene is absorbed into a solvent and passed into a reactor in which a catalyst and hydrogen are present. Under proper reactive conditions, the acetylene is converted into ethylene. The molar ratio of hydrogen to acetylene in the reactor is low, never exceeding approximately four. Additionally, the concentration of acetylene in solvent in the stream that is passed to the reactor is never less than 1%.

A byproduct of selective hydrogenation is C₄+ hydrocarbons (hydrocarbons with four or more carbon atoms). The C₄+ hydrocarbons are undesirable because they can accumulate on catalysts causing coke and fouling the catalyst. Additionally, the creation of the C₄+ hydrocarbons needlessly consumes the acetylene and can make ethylene separation from the rest of products more complicated.

Therefore, it would be desirable to have a process which reduces the production of the C₄+ hydrocarbons in a selective hydrogenation of acetylene to ethylene.

### SUMMARY OF THE INVENTION

It has been discovered that by reducing the concentration of acetylene in a stream passing into a hydrogenation reactor, the selectivity to C₄+ hydrocarbons is lowered and the selectivity to ethylene increases.

Therefore, a first embodiment of the invention may be characterized as a process for a liquid phase selective hydrogenation of acetylene to ethylene which includes: contacting acetylene from an acetylene rich stream with hydrogen in the presence of a catalyst under hydrogenation reaction conditions in a reaction zone to produce a reaction effluent; separating the reaction effluent in a separation zone into an overhead stream and a bottoms stream, the overhead stream being an ethylene rich stream; and, decreasing an amount of C₄+ hydrocarbons in the bottoms stream by decreasing a concentration of acetylene in at least a portion of the acetylene rich stream. The separation zone may be a hot separation zone.

A fraction of the bottoms stream from the separation zone may be combined with at least a portion of the acetylene rich stream. The acetylene rich stream and the bottoms stream from the separation zone preferably both include solvent.

The process may further include splitting the acetylene rich stream into at least two acetylene rich split streams and, injecting each acetylene rich split stream into the reaction zone. It is contemplated that the reaction zone comprises a reactor with at least two beds, each bed contains catalyst. A portion of acetylene rich stream is preferably injected into each bed the reaction zone. The acetylene rich stream may be split into unequal amounts.

The process may further include absorbing acetylene in a solvent and passing the mixture of acetylene and solvent to the reaction zone. In some embodiments, it is contemplated that the concentration of acetylene in the solvent passed to the reaction zone is less than 1.0 wt %.

A second embodiment of the invention may be characterized as a process for a liquid phase selective hydrogenation of acetylene to ethylene which includes: passing at least one stream comprising acetylene and solvent into a reaction zone; contacting acetylene with hydrogen in the presence of a catalyst under hydrogenation reaction conditions in the reaction zone; passing the reaction effluent from the reaction zone to a separation zone; separating the reaction effluent in the separation zone into an overhead stream and a bottoms stream, the overhead stream being an ethylene rich stream and the bottoms stream comprising solvent; and, combining a portion of the bottoms stream from the separation zone with the at least one stream being passed into the reaction zone.

It is contemplated that at least two streams, each comprising acetylene and solvent, are passed into the reaction zone. Each stream is passed into the reaction zone may be injected into the reaction zone at a different position.

In yet another embodiment, the present invention may be characterized as a process for decreasing a selectivity of C₄+ hydrocarbons in a liquid phase selective hydrogenation of acetylene to ethylene by: passing at least two streams into a reaction zone having a at least two beds, each stream comprising acetylene and solvent, and each bed including catalyst and receiving at least one stream being passed into the reaction zone; passing hydrogen in to the reaction zone; and, contacting hydrogen and acetylene in the presence of the catalyst under hydrogenation reaction conditions to produce a reaction effluent.

It is contemplated to separate the reaction effluent in the separation zone into an overhead stream and a bottoms stream. The overhead stream is an ethylene rich stream and the bottoms stream comprises solvent.

A portion of the bottoms stream from the separation zone may be combined with at least one of the at least two streams being passed into the reaction zone. It is also contemplated that each stream passed into the reaction zone receives a portion of the bottoms stream from the separation zone. A first stream passed into the reaction zone may receive a first amount of portion of the bottoms stream from the separation zone, and a second stream passed into the reaction zone may receive a second amount of portion of the bottoms stream from the separation zone. The second amount may be the same or different than the first amount.

In any of the embodiments of the present invention, a concentration of acetylene in the stream passed into the reaction zone may be less than 5 wt %, or between about 5 to about 1 wt %, or between about 3 to about 1 wt %, or between about 3 to 0.1 wt %, or between 2 to 0.5 wt % or less than 1.0 wt %.

These and other embodiments relating to the present invention should be apparent to those of ordinary skill in the art from the following detailed description of the invention.

### DETAILED DESCRIPTION OF THE DRAWINGS

The drawings are intended to be understood as an illustration of the present invention in which:

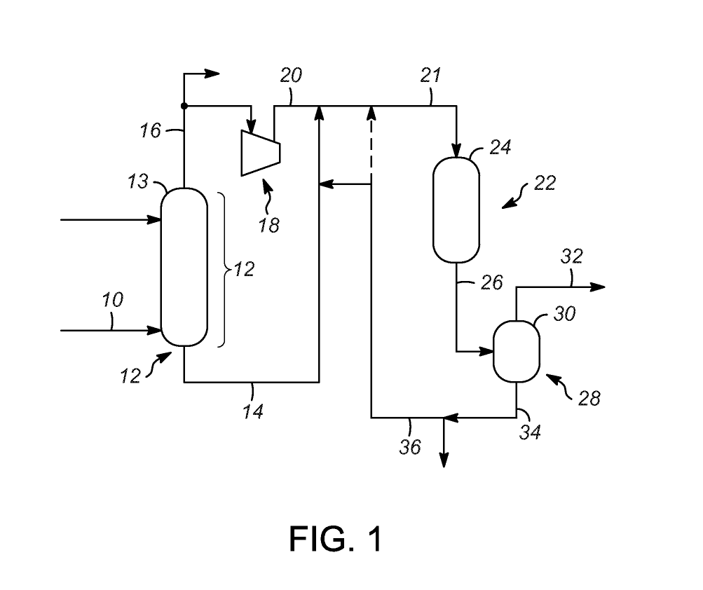

FIG. 1 shows a process flow diagram for the liquid phase selective hydrogenation of acetylene to ethylene according to one or more embodiments of the present invention; and,

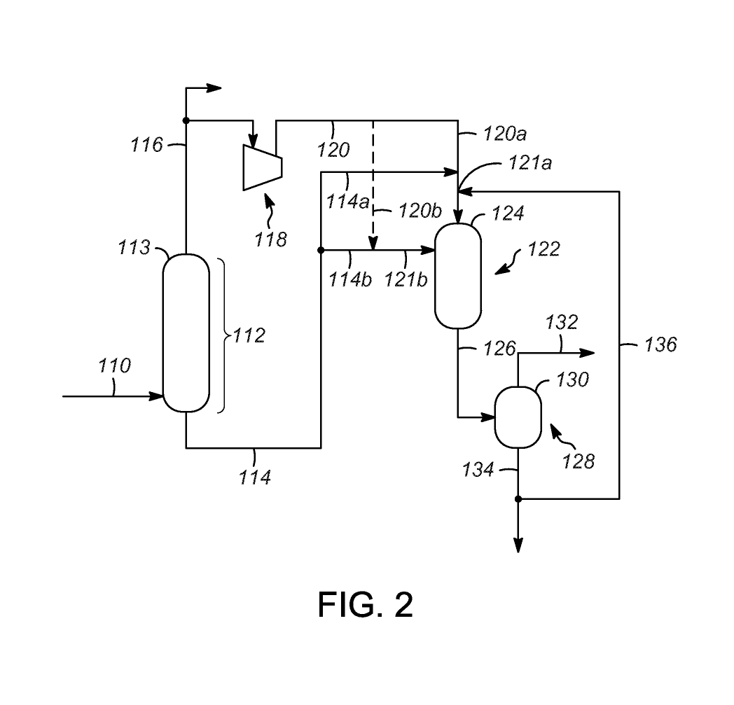

FIG. 2 shows a process flow diagram for the liquid phase selective hydrogenation of acetylene to ethylene according to one or more embodiments of the present invention.

### DETAILED DESCRIPTION OF THE INVENTION

As mentioned above, in a liquid phase selective hydrogenation of acetylene to ethylene according to the present invention, the concentration of acetylene in a stream passing into a hydrogenation reactor is decreased in order to lower the selectivity to C₄+ hydrocarbons and to increase the selectivity to ethylene. The concentration of acetylene (in solvent) may be less than 5 wt %, or between about 5 to about 1 wt %, or between about 3 to about 1 wt %, or between about 3 to 0.1 wt %, or between 2 to 0.5 wt %.

One process for providing the lower acetylene concentration is shown in FIG. 1, in which an acetylene rich vapor stream 10 containing acetylene may be passed to an absorption zone 12.

The acetylene containing stream 10 preferably is obtained from the pyrolysis of a hydrocarbon feed stream comprising methane for example natural gas; however, it is contemplated that the steam 10 may be obtained from any industrial process in which the effluent streams contain acetylene.

In the absorption zone 12, typically within an absorption column 13, acetylene is absorbed into a solvent, such as n-methyl-2-pyrrolidone (NMP), dimethylformamide (DMF), acetonitrile (ACN), and mixtures thereof. A first stream 14 being a liquid and comprising solvent and acetylene is removed from the absorption zone 12. A second stream 16 being a vapor stream that is lean in acetylene and comprising hydrogen gas is also removed from the absorption zone 12. In order to allow downstream reactors to operate at higher pressures, the second stream 16 (or a portion thereof) may be passed to a compression zone 18 to provide a compressed second stream 20.

The compressed second stream 20 and the first stream 14 from the absorption zone 12 are combined into a combined stream 21 which is passed to a hydrogenation zone 22. Additionally, carbon monoxide may be passed to the hydrogenation zone 22. While the second stream 16 from the absorption zone 12 may include carbon monoxide, carbon monoxide can also be recovered from a downstream reaction effluent stream or carbon monoxide may be added to the process from another source. The concentration of carbon monoxide in stream 16 may vary depending on the source of the acetylene rich stream 10 entering absorption zone 12. In an embodiment the carbon monoxide concentration will be in the range of about 1 to about 50 mol %, or about 5 to about 35 mol %, or about 5 to about 20 mol %.

The hydrogenation zone 22 may include at least one hydrogenation reactor 24. As is known, each hydrogenation reactor 24 includes a hydrogenation catalyst, typically a hydrogenation metal in an amount between 0.01 to 5.0 wt % on a support, wherein the hydrogenation metal is preferably selected from a Group VIII metal. Preferably the metal is platinum (Pt), palladium (Pd), nickel (Ni), or a mixture thereof. More preferably, a Group VIII metal is modified by one or more metals, selected from Group IB through IVA, such as zinc (Zn), indium (In), tin (Sn), lead (Pb), copper (Cu), silver (Ag), gold (Au) in an amount between 0.01 and 5 wt. %. Preferred supports are aluminum oxides (aluminas), pure or doped with other metal oxides, synthetic or natural (i.e. clays). More preferred supports are Alpha-Aluminas of various shape and size (i.e. spheres, extrudates), with high degree of conversion to Alpha phase.

In the hydrogenation reactor 24, in the presence of the catalyst, under hydrogenation conditions, the hydrogen reacts with the acetylene to preferably produce ethylene. The hydrogen may be in the second stream 16 from the absorption zone 12, or hydrogen may come from a portion of a downstream reaction effluent, or hydrogen may be added to the process.

Typical hydrogenation reaction conditions in the hydrogenation reactor 24 include a temperature that may range between 50° C. and 250° C., preferably between 100° C. to 200° C. Additionally, the hydrogenation reactor 24 is operated at a high pressure which may range between approximately 0.69 MPa (100 psig) and 3.4 MPa (500 psig), preferably between approximately 1.0 MPa (150 psig) and 2.8 MPa (400 psig). The liquid hour space velocity (LHSV) at the reactor inlet of the hydrogenation reaction can range between 1 and 100 h⁻¹, with preferred ranges being between 5 and 50 h⁻¹, between 5 and 25 h⁻¹, and between 5 and 15 h⁻¹.

The products of the hydrogenation reaction can be recovered from the hydrogenation reactor 24 via a stream 26. The reactor effluent stream 26 is passed to a separation zone 28 which contains, for example, a separator vessel 30. In one embodiment, the separation zone 28 is a hot separation zone operating at nominally the same temperature as the reactor hydrogenation reactor 24. By “hot separation zone” it is meant that the reaction effluents are not actively cooled before being passed to the separation zone (the effluent may lose some amount of heat in the transfer process). In another embodiment the reactor effluent stream 26 may be cooled prior to entering separation zone 28 to decrease the temperature to achieve the desired compositions in the vapor stream 32 and liquid stream 30. This would be particularly desirable if the hydrogenation reactor is operated at relatively low conversion, for example less than about 95%.

In the separator vessel 30 of the separation zone 28, the reaction effluents are separated into an overhead vapor stream 32 and a bottoms liquid stream 34. The overhead vapor stream 32 is rich in ethylene and may contain other gases. This stream 32 may be passed to other processing and separation zones, the particulars of which are not necessary for an understanding and practicing of the present invention. Additionally, since this stream may include carbon monoxide and hydrogen, a portion of this stream 32 may be recycled back to the stream 21 entering the hydrogenation zone 22 to provide carbon monoxide and hydrogen for the hydrogenation reactions.

The bottoms liquid stream 34 is rich in solvent. This stream 34 may be removed from the separation zone 28 and passed to further processing zones as well. The further processing may include separation zones to remove byproducts produced in the hydrogenation reactor 24 such as C₄+ hydrocarbons or water from at least a portion of the circulating solvent. This separation may also be necessary to enable recycle of the solvent to absorption zone 12. One skilled in the art will appreciate that the cost and frequency of such separations will be decreased by minimizing byproducts such as C₄+ hydrocarbons in the hydrogenation reactor 24.

As shown in FIG. 1, in various embodiments of the present invention, at least a portion 36 of this stream 34 is split and recycled with the first stream 14 from the absorption zone 12 or with a stream 21 entering the hydrogenation zone 22. By recycling at least the portion 36 of the solvent rich stream 34, the concentration of acetylene in solvent entering the hydrogenation zone 22 will be decreased.

Additionally, the recycling of solvent will also de-couple the reactor inlet(s) acetylene concentration from the upstream absorption zone 12 conditions. This is advantageous because it allows the absorption zone 12 conditions to be optimized to provide a more efficient, less costly process design in terms of energy efficiency and equipment cost. If the conditions in the absorber and hydrogenation reactor are coupled then the total solvent circulation rate and solvent inventory of the process may be higher when compared to the proposed flow scheme in which provides a short recycle path to the hydrogenation reactor inlet where it is desirable to reduce the acetylene concentration and allows the acetylene concentration in the solvent to be maximized in the absorption zone 12. An additional advantage of this recycle path 36 is that the equipment in the absorption zone 12 will be smaller and therefore less costly than if the concentration of acetylene in the solvent were decreased by increasing the solvent circulation rate to the absorption zone 12. The solvent flow to the absorption zone 12 may be about 2 to about 10 times lower with the proposed flow scheme than if the concentration of acetylene to the hydrogenation reactor 24 were to be controlled by adjusting the conditions in absorption zone 12.

The lowering of the acetylene concentration can be appreciated in the following example in which an acetylene concentration in a stream from an absorption zone is 2 wt % in solvent (at the hydrogenation reactor inlet). By recycling solvent that has been separated from the hydrogenation reactor effluent at a recycle to feed ratio of 1, the concentration of acetylene in the stream from the absorption zone will be reduced to 1 wt %. As the recycle to feed ratio is increased, the concentration will be reduced further, for example with a recycle to feed ratio of 10, the concentration will be 0.2 wt %. Thus, the use of the solvent recycle provides an effective way to lower the acetylene concentration of a stream entering a hydrogenation reactor.

In another embodiment of the present invention, the acetylene concentration in the solvent is lowered through the use of a split feed injection.

As shown in FIG. 2, an acetylene rich vapor stream 110 comprising acetylene is obtained from any industrial process discussed above in which acetylene is produced. A preferred source of the acetylene rich vapor is from the pyrolysis of a stream comprising methane such as natural gas which will also include for example hydrogen and carbon monoxide.

The acetylene rich vapor stream 110 is passed to an absorption zone 112 in which, in a column 113, acetylene is absorbed into a solvent, such as NMP, DMF, ACN, and mixtures thereof. A first steam 114 comprising solvent and acetylene is recovered from the absorption zone 112. A second stream 116 comprising hydrogen gas and carbon monoxide is also recovered from the absorption zone 112.

The second stream 116 is passed to a compression zone 118 to provide a compressed second stream 120 so that downstream reactors may be run at higher pressures. The compressed second stream 120 may be directed to the hydrogenation reactor 124 in one or more locations.

For example in an embodiment the entirety of stream 120 may be combined with stream 114a to form combined feed stream 121a. In another embodiment stream 120 may be split into a least two portions 120a, 120b, each portion to combine with a portion 114a, 114b of the first stream 114 to form, for example, combined feed streams 121a and 121b which are passed to hydrogenation zone 122 as described below.

The hydrogenation zone 122 includes at least one hydrogenation reactor 124. The hydrogenation reactor 124 is described above with respect to the first embodiment, the description of which is incorporated herein by reference.

As mentioned above, in this embodiment the concentration of acetylene in the solvent is diluted through the use of a split feed injection into the hydrogenation reactor 124. Thus, the liquid feed stream 114 passed into the hydrogenation reactor 124 is split into a plurality of streams 114a, 114b. The split streams 114a, 114b may be of equal amounts or they may be different. Since the hydrogenation reactor 124 may contain a plurality of beds at different vertical levels, each bed may receive a split stream 114a, 114b. With the split stream injection, the solvent in the streams injected into the reactor beds toward the top of the reactor 124 will flow downward in the reactor 124. This will dilute the acetylene concentration in the reactor beds towards the bottom of the reactor 124.

The affect the split stream injection on the acetylene concentration will be appreciated based upon the following example for a stream having a concentration of 3 wt % acetylene. If the stream is split into two equal portions and injected into two beds, the concentration of acetylene at the first, or top-most bed will be 3 wt %. However, the concentration of acetylene at the second bed, lower bed, will be 1.5 wt % because the solvent from the first bed will be substantially depleted of acetylene and will flow downward and dilute the acetylene in the stream flowing into the second bed. The stream is typically considered as substantially depleted of acetylene when the concentration has been reduced by about 90% relative to stream 114 from the absorption zone 112. Thus, a lower concentration can be achieved by increasing the number of beds and split streams used.

In a most preferred embodiment, the use of a spilt feed injection is coupled with the use of a recycled solvent stream described above with respect to FIG. 1. Thus, as shown in FIG. 2, a stream of reactor effluent 126 may be passed to a separation zone 128 which contains a separator vessel 130. It is preferred, although not required, that the separation zone 128 is a hot separation zone, meaning it is operated at a temperature nominally equal to the reactor effluent. In another embodiment the reactor effluent stream may be cooled prior to entering separation zone 128 or within separation zone 128.

In the separator vessel 130 of the separation zone 128, the reactor effluent is separated into an overhead vapor stream 132 and a bottoms liquid stream 134. The overhead vapor stream 132 is rich in ethylene and may contain other gases. This stream 132 may be passed to other processing and separation zones, the particulars of which are not necessary for an understanding and practicing of the present invention. Like the first embodiment, this stream 132, or a portion thereof, may be recycled to provide hydrogen, carbon monoxide, or both to the hydrogenation zone 122.

The bottoms liquid stream 134 is rich in solvent and substantially depleted of acetylene. This stream 134 may be removed from the separation zone 128 and passed to further processing zones as well for example to remove C₄+ hydrocarbons and water as described above. Similar to the first embodiment of the present invention, at least a portion 136 of this stream 134 may be split and recycled to the stream 121a entering the first catalyst bed in the hydrogenation zone 122.

The use of both the recycle solvent and the split feed injection will allow for a smaller recycle to feed ratio to be used to achieve the lower concentrations. For example, for the exemplary stream discussed above for the first embodiment (having a 2 wt % acetylene concentration), if the stream was split into two equal parts, only a 5:1 recycle ratio would be needed to achieve a 0.2 wt % acetylene (as opposed to the 10:1 recycle ratio needed without the use of the split feed). Thus, by using the recycle stream and splitting the stream into two equal split streams and injecting each split stream in to a catalyst bed in vertical series, the recycle to feed ratio can be reduced by 50% to achieve about 0.2 wt % acetylene at the inlet of each bed.

In order to demonstrate the benefits of the lower acetylene concentration on the hydrogenation reactions, experimental data on acetylene hydrogenation in liquid NMP solvent were collected. For each experiment, a bimetallic Pd—Ag/Alpha-Al catalyst consisting of Pd and Ag on an alpha alumina support was used with a stream having a flow rate of LHSV 10 h⁻¹ (on NMP basis). The reactions conditions included a pressure of 1.72 MPa (250 psig) and a hydrogen to acetylene molar ratio of 1.5 and a hydrogen to carbon monoxide molar ratio of 2. The temperature was approximately 140° C. for all of the experiments in an attempt to maintain acetylene conversion between approximately 90 to 95%. The results of the experiments are shown below in Table 1.

TABLE 1

| | Exp. 1 | Exp. 2 | Exp. 3 | Exp. 4 |
|---|---|---|---|---|
| C₂H₂ in NMP (wt %) | 1.5 | 1.5 | 0.9 | 0.45 |
| Conversion (wt %) | 94.7 | 89.5 | 96.3 | 87.4 |
| Selectivity (wt %) | | | | |
| Ethylene | 90.5 | 89.9 | 93.5 | 95.5 |
| Ethane | 0.14 | 0.28 | 0.69 | 1.8 |
| C₃ oxygenates | ~2.4 | 2.2 | 1.8 | 1.2 |
| C₄+ (wt %) | 6.6 | 6.8 | 3.8 | 1.3 |

While there are some differences in catalyst age between the experimental data, the overall trend towards lower C₄+ hydrocarbon selectivity and yield as a result of lowering acetylene concentration in NMP is clear. This trend is particularly clear when the C₄+ hydrocarbon selectivity in experiments 1 and experiments 3 are compared. In each of these experiments the acetylene conversion was approximately 95%. It can be appreciated that decreasing the acetylene concentration from 1.5 wt % in experiment 1 to 0.9 wt % in experiment 3 resulted in higher ethylene selectivity and lower C₄+ hydrocarbon selectivity. A similar impact is seen when experiments 2 and 4 are compared at slightly lower conversion. Thus, it can be concluded from this data that lower acetylene concentration in the solvent favors the production of the desired product ethylene and decreases the undesired byproducts such as C₄+ hydrocarbons and oxygenates. While not intending to be bound to any particular theory, it is believed that this result is because of the bimolecular nature of oligomerization reactions.

Therefore, one or more embodiments of the present invention provide a process which decreases the both C₄+ hydrocarbon and oxygenate selectivity in a selective hydrogenation of acetylene to ethylene. This will allow for better recovery of the desired products, better utilization of the acetylene, and less production of undesirable components.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for a liquid phase selective hydrogenation of acetylene to ethylene comprising:
contacting acetylene from an acetylene rich stream with hydrogen in the presence of a catalyst under hydrogenation reaction conditions in a reaction zone to produce a reaction effluent;
separating the reaction effluent in a separation zone into an overhead stream and a bottoms stream, the overhead stream being an ethylene rich stream; and,
decreasing an amount of C₄+ hydrocarbons in the bottoms stream by decreasing a concentration of acetylene in at least a portion of the acetylene rich stream.

2. The process of claim 1 further comprising:
combining a fraction of the bottoms stream from the separation zone with at least a portion of the acetylene rich stream.

3. The process of claim 2 wherein the acetylene rich stream includes solvent and wherein the bottoms stream from the separation zone includes solvent.

4. The process of claim 1 further comprising:
splitting the acetylene rich stream into at least two acetylene rich split streams; and,
injecting each acetylene rich split stream into the reaction zone.

5. The process of claim 4 wherein the reaction zone comprises a reactor with at least two beds, each bed containing catalyst and further comprising:
injecting a portion of acetylene rich stream into each bed of the reaction zone.

6. The process of claim 4, wherein the acetylene rich stream are split into unequal amounts.

7. The process of claim 4, further comprising:
combining a fraction of the bottoms stream with at least a portion of the acetylene rich stream.

8. The process of claim 7 wherein the acetylene rich stream includes solvent and wherein the bottoms stream from the separation zone includes solvent.

9. The process of claim 1 further comprising:
absorbing acetylene in a solvent; and,
passing the mixture of acetylene and solvent to the reaction zone.

10. The process of claim 9 wherein the concentration of acetylene in the solvent passed to the reaction zone is less than 1.0 wt %.

11. A process for decreasing a selectivity of C₄+ hydrocarbons in a liquid phase selective hydrogenation of acetylene to ethylene comprising:
passing at least one stream comprising acetylene and solvent into a reaction zone;
contacting acetylene with hydrogen in the presence of a catalyst under hydrogenation reaction conditions in the reaction zone;
passing the reaction effluent from the reaction zone to a separation zone;
separating the reaction effluent in the separation zone into an overhead stream and a bottoms stream, the overhead stream being an ethylene rich stream and the bottoms stream comprising solvent; and,
combining a portion of the bottoms stream from the separation zone with the at least one stream being passed into the reaction zone.

12. The process of claim 11 wherein a concentration of acetylene in the stream passed into the reaction zone is less than 5 wt %.

13. The process of claim 11 wherein a concentration of acetylene in the stream passed into the reaction zone is between about 0.1 to about 3 wt %.

14. The process of claim 11 wherein the step of passing at least one stream comprising acetylene and solvent into a reaction zone comprises:
passing at least two streams into the reaction zone, each stream comprising acetylene and solvent.

15. The process of claim 14 wherein each stream is passed into the reaction zone at a different position.

16. A process for decreasing a selectivity of C₄+ hydrocarbons in a liquid phase selective hydrogenation of acetylene to ethylene comprising:
passing at least two streams into a reaction zone having a at least two beds, each stream comprising acetylene and solvent, and each bed including catalyst and receiving at least one stream being passed into the reaction zone;
passing hydrogen in to the reaction zone; and,
contacting hydrogen and acetylene in the presence of the catalyst under hydrogenation reaction conditions to produce a reaction effluent.

17. The process of claim 16 further comprising:
separating the reaction effluent in the separation zone into an overhead stream and a bottoms stream, the overhead stream being an ethylene rich stream and the bottoms stream comprising solvent; and,
combining a portion of the bottoms stream from the separation zone with at least one of the at least two streams being passed into the reaction zone.

18. The process of claim 17 wherein each stream passed into the reaction zone receives a portion of the bottoms stream from the separation zone.

19. The process of claim 18 wherein a first stream being passed into the reaction zone receives a first amount of portion of the bottoms stream from the separation zone, and wherein a second stream being passed into the reaction zone receives a second amount of portion of the bottoms stream from the separation zone, the second amount being different than the first amount.

20. The process of claim 19 wherein a concentration of acetylene in the stream passed into the reaction zone is between about 0.1 to about 5 wt %.
